(19) United States
(12) Patent Application Publication
(10) Pub. No.: US 2017/0105979 A1
(43) Pub. Date: Apr. 20, 2017

(54) USE OF A TORASEMIDE-BASED VETERINARY COMPOSITION FOR LOW-DOSE ADMINISTERING

(71) Applicant: VIRBAC, CARROS CEDEX (FR)

(72) Inventors: Pierre-Louis TOUTAIN, La Salvetat Saint Gilles (FR); Jean-Pascal MARC, Saint Paul De Vence (FR)

(73) Assignee: VIRBAC, CARROS CEDEX (FR)

(21) Appl. No.: 15/127,195

(22) PCT Filed: Mar. 19, 2015
(86) PCT No.: PCT/IB2015/052012

§ 371 Date: Sep. 19, 2016

(30) Foreign Application Priority Data
Mar. 19, 2014 (FR) 1452258

(51) Int. Cl. A61K 31/44; A61K 45/06; A61K 9/20

(52) U.S. Cl. CPC A61K 31/44; A61K 9/20; A61K 45/06

(57) ABSTRACT

The present invention relates to a veterinary pharmaceutical composition containing torasemide to be used for treating cardiac failure. The torasemide is administered in a daily dose of 0.02 mg/kg to 0.1 mg/kg during long-term treatment.

The present invention relates to compositions intended for treating animals suffering from heart failure, said compositions comprising torasemide.

### PRIOR ART

Torasemide is a loop diuretic which acts on the ascending part of the loop of Henley of the kidney. This molecule belongs to the pyridine-sulfonylurea class and is used in the treatment of edema associated with heart failure, and of kidney diseases and in the treatment of hypertension, in human and veterinary therapy.

Heart failure represents a serious disease in dogs, which affects up to 10% of the overall canine population. While this ailment remains complex and serious, knowledge of its physiopathology has progressed over the past few years, making its diagnosis easier, and enabling new therapeutics to emerge. The prognosis thereof has greatly improved and it is now not rare to see dogs survive several years with heart failure.

In congestive heart failure, “compensatory” mechanisms make it possible to maintain a sufficient cardiac output and a sufficient tissue perfusion during the first phase of the disease, during which there is no clinical symptom; the heart failure is termed asymptomatic or compensated. When the compensatory mechanisms are exceeded, the first clinical symptoms appear: this is the decompensated or symptomatic heart failure phase.

At this stage, the decrease in cardiac output leads to a decrease in renal perfusion which in turn leads to the secretion of renin and also arterial and venous vasoconstriction. In a second step, aldosterone secretion is stimulated, this hormone acting on the distal convoluted tube by increasing sodium reabsorption and potassium elimination. The resulting plasma hypertonicity stimulates the reabsorption of water which increases volemia. Aldosterone also has a direct vasoconstrictive activity. Finally, plasma hypertonicity leads to the release of anti-diuretic hormone (ADH), which promotes water retention. All these mechanisms result in vasoconstriction and in hypervolemia, the latter possibly being likened to an excessively high sodium level in the body. This condition is treated by administering diuretics, making it possible to increase the excretion of ions, in particular sodium (Na⁺) ions, in the urine.

Several stages of the disease can be identified. Heart failure can be categorized according to the NYHA or ISACHC classification (see table 1). For example, the asymptomatic phase of heart failure corresponds to class I of the ISACHC classification, and classes II and III correspond to stages at which the disease is symptomatic.

TABLE 1
ISACHC classification

| Asymptomatic stage | Class I | Compensated cardiopathy | Ia - absence of cavity dilatation (no |
|---|---|---|---|
| | | No clinical signs | cardiac modification visible on x-ray or |
| | | Signs of cardiopathy detectable on | echo) |
| | | examination (murmur, cardiomegaly, | IIb - cavity dilatation (ex: left atrial |
| | | etc.) | dilatation or cardiomegaly) |
| Symptomatic stage: | Class II | Slight or moderate heart failure | |
| congestive heart failure | | Congestive clinical symptoms after exercise, affecting quality of life | |
| | Class III | Advanced or severe heart failure | IIIa - Possible care at home |
| | | Permanent serious clinical symptoms, | IIIb - Hospitalization and emergency |
| | | even at rest | care required (acute lung edema) |
| | | Exercise impossible | |

Currently, the reference treatment for congestive heart failure in animals is the administration of furosemide, a loop diuretic, at daily doses of between 2 mg and 5 mg/kg/day. Torasemide is considered to be an equivalent of furosemide. Several studies have compared the effects of these two diuretics, on healthy dogs and on dogs suffering from congestive heart failure.

Ghys et al. (Drug Research, 1985) have compared the effects of torasemide and of furosemide on healthy rats and dogs, and have demonstrated a relationship between the dose administered and the size of the diuretic effect and of the ion secretion. The effects measured are done so following a single administration, and not during prolonged treatments.

Hori et al. (AJVR, 2007) have shown on healthy dogs that, during prolonged treatments of 14 days, the dogs treated with furosemide develop “diuretic resistance”, whereas this resistance does not occur in dogs treated with torasemide, administered at 0.4 mg/kg/day. Unfortunately, these two compounds lead to an increase in plasma creatinine concentrations.

Uechi et al. (J. Vet. Med. Sci. 2003) have studied the diuretic effects of furosemide and of torasemide on healthy and sick dogs, over a period of 7 days. The diuretic effect of furosemide is rapid, visible approximately 1 hour after administration, but decreases 6 hours after administration. Conversely, the effect of torasemide, administered at 0.2 mg/kg, appears only 2 to 4 hours after administration and persists for 12 hours in the treated animals. After 7 days, potassium excretion in the urine in the sick dogs treated with torasemide is less than that observed for the dogs treated with furosemide.

Caro-Vadillo et al. (Veterinary Record, 2007) have measured, in dogs suffering from congestive heart failure, the effect of torasemide administered at 0.2 mg/kg/day in a dose given in the morning for 28 days, on the blood and urine levels of sodium, potassium, chloride ions, calcium, phosphorus and magnesium. They have shown that, in the treated dogs, potassium excretion in the urine is increased, but that sodium excretion is not increased.

Peddle et al. (J. Vet. Cardiology, 2012) have tested, in dogs suffering from congestive heart failure in the stable phase, the oral twice-daily administration of furosemide (5.13 mg/kg/day) or of torasemide (0.5 mg/kg/day) for 7 days. The two compounds made it possible to maintain the disease in a stable state from a clinical point of view; the authors nevertheless recommend favoring torasemide for future studies, since the latter has a better diuretic effect.

Thus, even though furosemide remains the reference treatment, there is an increasing interest in the use of torasemide in the treatment of cardiac ailments in animals, as is presented in patent application EP 2 514 421.

The good results in favor of the use of torasemide should not, however, mean that its main harmful effect is forgotten, said effect being the exaggerated increase in diuresis and therefore in the excretion of ions, in particular sodium and potassium ions, this creating, if the diuresis is too marked, an ion imbalance in the body of the treated animal. This ion imbalance leads to other harmful effects such as drops in blood pressure, increased plasma aldosterone and creatinine concentration, and increased water consumption, these effects being all the more marked if the treatment is extended over long periods.

Novel therapeutic compositions are therefore actively sought in order to overcome the harmful side effects of treatments using loop diuretics. The question is all the more important when the animals treated are elderly animals.

### SUMMARY OF THE INVENTION

The inventors of the present application have developed a novel dosage of torasemide, which makes it possible to maintain the beneficial effects of this treatment while at the same time significantly reducing the harmful effects described above.

Surprisingly, the inventors show here that low doses of torasemide, less than or equal to 0.1 mg/kg/day, make it possible to maintain the beneficial effects observed during treatments with higher dosages.

In particular, the present invention relates to a veterinary pharmaceutical composition comprising torasemide, for use thereof in the treatment of heart failure, the torasemide being administered at a daily dosage of between 0.02 mg/kg and 0.1 mg/kg during a long-term treatment.

This composition is particularly suitable for the treatment of cats or dogs suffering from heart failure, at all stages of the disease, including during the asymptomatic phase, then during the decompensation phase, and in particular when the cats or dogs are elderly.

### DESCRIPTION OF THE FIGURES

For all the figures, the “check” pattern represents the values obtained without treatment; the “horizontal stripes” pattern represents the values obtained with treatment at 0.05 mg/kg of torasemide; the “plain white” pattern represents the values obtained with treatment at 0.5 mg/kg of torasemide; the “plain gray” pattern represents the values obtained with treatment of 5 mg/kg of furosemide.

FIG. 1. Mean (±standard deviation) of urinary sodium clearance expressed in ml/h (y-axis) between 0-24 h without treatment (column 1), after a single administration of torasemide at 0.05 mg/kg (column 2) or after a single administration of torasemide at 0.5 mg/kg (column 3) in female beagle dogs (n=6 dogs per group).

FIG. 2. Mean (±standard deviation) of the urinary sodium clearance expressed in ml/h (y-axis) between 0-24 h after repeated administrations of torasemide at 0.05 mg/kg/day, of torasemide at 0.5 mg/kg/day or of furosemide at 5 mg/kg/day on D-7 (before administration), D7 (first administration), D18 (twelfth administration) and D28 (twenty-second and final administration).

FIG. 3. Mean (±standard deviation) of the urine volume expressed in ml (y-axis) between 0-24 h after repeated administrations of torasemide at 0.05 mg/kg/day, of torasemide at 0.5 mg/kg/day or of furosemide at 5 mg/kg/day on D-7 (before administration), D7 (first administration), D18 (twelfth administration) and D28 (twenty-second and final administration).

FIG. 4. Mean (± standard deviation) of the volume of water absorbed, expressed in ml (y-axis), between 0-24 h after repeated administrations of torasemide at 0.05 mg/kg/day, of torasemide at 0.5 mg/kg/day or of furosemide at 5 mg/kg/day on D-7 (before administration), D7 (first administration), D18 (twelfth administration) and D28 (twenty-second and final administration).

FIG. 5. Mean (±standard deviation) of the urinary potassium clearance expressed in ml/h (y-axis) between 0-24 h after repeated administrations of torasemide at 0.05 mg/kg/day, of torasemide at 0.5 mg/kg/day or of furosemide at 5 mg/kg/day on D-7 (before administration), D7 (first administration), D18 (twelfth administration) and D28 (twenty-second and final administration).

FIG. 6. Mean (±standard deviation) of the urinary creatinine clearance expressed in ml/min (y-axis) between 0-24 h after repeated administrations of torasemide at 0.05 mg/kg/day, of torasemide at 0.5 mg/kg/day or of furosemide at 5 mg/kg/day on D-7 (before administration), D7 (first administration), D18 (twelfth administration) and D28 (twenty-second and final administration).

FIG. 7. Mean (±standard deviation) of the plasma urea concentration expressed in mmol/l (y-axis) between 0-24 h after repeated administrations of torasemide at 0.05 mg/kg/day, of torasemide at 0.5 mg/kg/day or of furosemide at 5 mg/kg/day on D-7 (before administration), D7 (first administration), D18 (twelfth administration) and D28 (twenty-second and final administration).

FIG. 8. Mean (±standard deviation) of the plasma aldosterone concentration expressed in pg/ml (y-axis) between 0-24 h after repeated administrations of torasemide at 0.05 mg/kg/day, of torasemide at 0.5 mg/kg/day or of furosemide at 5 mg/kg/day on D-7 (before administration), D7 (first administration), D18 (twelfth administration) and D28 (twenty-second and final administration).

FIG. 9. Mean (±standard deviation) of the urinary fractional excretion of sodium expressed as % (y-axis) between 0-24 h without treatment (1) or after a single administration of torasemide at 0.05 mg/kg (2) or of torasemide at 0.5 mg/kg (3) in female beagle dogs.

FIG. 10. Mean (±standard deviation) of the urinary fractional excretion of sodium expressed as % (y-axis) between 0-24 h after repeated administrations of torasemide at 0.05 mg/kg/day, of torasemide at 0.5 mg/kg/day or of furosemide at 5 mg/kg/day on D-7 (before administration), D7 (first administration), D18 (twelfth administration) and D28 (twenty-second and final administration).

### DETAILED DESCRIPTION OF THE INVENTION

The present invention relates to a veterinary pharmaceutical kit for use thereof in the treatment and/or prevention of heart failure, comprising a plurality of dosage units, each dosage unit comprising torasemide, and being suitable for oral administration of torasemide in a daily dosage of from 0.02 to 0.1 mg/kg during a long-term treatment.

The present invention also relates to a composition comprising torasemide, for use thereof as a veterinary medicament intended for treating and/or preventing heart failure in mammals, the torasemide being administered at a daily dosage of between 0.02 mg/kg and 0.1 mg/kg during a long-term treatment.

The present invention thus relates to a veterinary kit or to a veterinary composition, in particular for use in domestic animals, in particular domestic mammals, and more particularly cats, dogs, hamsters, rabbits, guinea pigs, ferrets, and other mammalian species listed in the decree of Aug. 11, 2006, emanating from the French ministry of ecology and sustainable development.

The term “veterinary pharmaceutical kit” denotes a kit comprising:
dosage units as defined below, comprising pharmaceutical compositions for veterinary use, and
optionally, a user information leaflet, specifying the animal for which the dosage units are intended, and the dosage regime, in particular as a function of the weight of the animal,
optionally, packaging suitable for the packaging of the dosage units.

The term “heart failure” denotes the inability of the heart to meet the metabolic needs of the organs, i.e. to provide a perfusion pressure that is sufficient to ensure the diffusion of oxygen and of nutrients from the blood to the tissues. It manifests itself in particular when a congenital or acquired cardiopathy impairs cardiac performance.

According to the invention, the “treatment of heart failure” begins as soon as heart failure is clinically observed, even if the symptoms are not yet apparent during the first phase, termed “asymptomatic” or “compensated”, of the disease (ISACHC class I). The term “prevention of heart failure” is intended to mean the administration of torasemide, at a dose according to the invention, to animals that have not been diagnosed as suffering from heart failure, but that exhibit risks, in particular genetic risks, of developing heart failure at a time in their life. The invention also makes it possible to treat “symptomatic” animals (ISACHC class II or III).

The term “dosage unit” denotes any practical packaging which allows the user, the owner of the domestic animal or the veterinarian, to administer to an animal the desired daily dose of torasemide, of from 0.02 mg/kg to 0.1 mg/kg.

The term “daily dosage” denotes the amount of active ingredient administered to the animal over a period of 24 h.

The term “torasemide” denotes the molecule of formula: N-[(isopropyl-amino)carbonyl]-4-[(3-methylphenyl)amino]pyridine-3-sulfonamide, of CAS number 56211-40-6.

The expression “long-term treatment” denotes a treatment of at least seven days, comprising the administration of a daily dose of torasemide at the doses indicated in the present application, in one or more intakes. In particular, the treatment may last at least 14 days, at least 21 days, at least 28 days, at least 30 days, at least one month, two months, three months, four months, five months, six months, seven months, eight months, nine months, ten months, eleven months, twelve months or several years.

One of the main advantages of the invention lies in the fact that the torasemide is administered at daily doses that are lower than usual, this making it possible to prolong its administration over longer periods of treatment, in particular of at least 7 days, while at the same time minimizing the side effects.

According to one preferred aspect of the invention, the torasemide is administered to animals suffering from stage I, II or IIIa heart failure, in any event to animals in which the disease has not reached a stage that is too severe and/or irreversible.

According to one particular aspect of the invention, in the pharmaceutical kit, the dosage units are suitable for the administration of a daily dose of torasemide of less than 0.1 mg/kg. Likewise, the pharmaceutical composition is suitable for the administration of a daily dosage of torasemide of less than 0.10 mg/kg. This dosage is particularly indicated for the treatment of dogs and cats.

According to one particular aspect of the invention, the torasemide is administered at a daily dosage of between 0.02 mg/kg and 0.09 mg/kg during a long-term treatment. According to another aspect, the torasemide is administered at a daily dosage of between 0.03 mg/kg and 0.08 mg/kg during a long-term treatment. According to another aspect, the torasemide is administered at a daily dosage of between 0.04 mg/kg and 0.06 mg/kg during a long-term treatment. These dosages are particularly indicated for the treatment of dogs and cats.

According to one particular aspect of the invention, in the pharmaceutical kit, the dosage units are suitable for the administration of a daily dosage of torasemide of approximately 0.05 mg/kg. Likewise, the pharmaceutical composition is suitable for the administration of a daily dosage of torasemide of approximately 0.05 mg/kg.

According to one particular aspect of the invention, the torasemide is administered at a daily dosage of approximately 0.050 mg/kg. This dosage is particularly indicated for the treatment of dogs and cats.

According to another particular aspect of the invention, the pharmaceutical kit is characterized in that each daily dosage unit comprises two subunits for an administration in two daily intakes. Likewise, the pharmaceutical composition may be administered in two daily intakes.

This is because the samples taken 12 hours after the administration of torasemide show that the diuretic effect begins to decrease between 12 and 24 hours post-administration; it may therefore be judicious to administer the composition twice-daily, in order to maintain the maximum effect over a longer period of time.

In particular, the pharmaceutical kit as defined above has dosage units, each comprising an amount of torasemide ranging from 0.02 mg (animal of 2 kg×2 daily intakes) to 5 mg (animal of 50 kg×1 daily intake). Each kit will be suitable for the weight of the animal to be treated; various dosages particularly suitable for dogs and cats are thus illustrated below:

Daily Dosage of 0.02 mg/kg:

For an animal of 2 kg, the daily intake will have to be 0.04 mg/day. The kit will thus comprise dosage units comprising 0.04 mg for the purpose of a daily administration, and 0.02 mg for the purpose of a twice-daily administration.

For an animal of 5 kg, the daily intake will have to be 0.1 mg/day. The kit will thus comprise dosage units comprising 0.1 mg for the purpose of a daily administration, and 0.05 mg for the purpose of a twice-daily administration.

For an animal of 10 kg, the daily intake will have to be 0.2 mg/day. The kit will thus comprise dosage units comprising 0.2 mg for the purpose of a daily administration, and 0.1 mg for the purpose of a twice-daily administration.

For an animal of 50 kg, the daily intake will have to be 1 mg/day. The kit will thus comprise dosage units comprising 1 mg for the purpose of a daily administration, and 0.5 mg for the purpose of a twice-daily administration.

Daily Dosage of 0.1 mg/kg:

For an animal having a weight of 2 kg, the daily intake will have to be 0.2 mg/day. The kit will thus comprise dosage units comprising 0.2 mg for the purpose of a daily administration, and 0.1 mg for the purpose of a twice-daily administration.

For an animal of 5 kg, the daily intake will have to be 0.5 mg/day. The kit will thus comprise dosage units comprising 0.5 mg for the purpose of a daily administration, and 0.25 mg for the purpose of a twice-daily administration.

For an animal of 10 kg, the daily intake will have to be 1 mg/day. The kit will thus comprise dosage units comprising 1 mg for the purpose of a daily dosage, and 0.5 mg for the purpose of a twice-daily administration.

For an animal of 50 kg, the daily intake will have to be 5 mg/day. The kit will thus comprise dosage units comprising 5 mg for the purpose of a daily administration, and 2.5 mg for the purpose of a twice-daily administration.

Preferably, the pharmaceutical kit as defined above comprises dosage units suitable for an administration of 0.05 mg/day of torasemide, i.e. units comprising an amount of torasemide ranging from 0.05 mg to 2.5 mg.

Daily Dosage of 0.05 mg/kg:

For an animal having a weight of 2 kg, the daily intake will have to be 0.1 mg/day. The kit will thus comprise dosage units comprising 0.1 mg for the purpose of a daily administration, and 0.05 mg for the purpose of a twice-daily administration.

For an animal of 5 kg, the daily dosage will have to be 0.25 mg/day. The kit will thus comprise dosage units comprising 0.25 mg for the purpose of a daily administration, and 0.125 mg for the purpose of a twice-daily administration.

For an animal of 10 kg, the daily intake will have to be 0.5 mg/day. The kit will thus comprise dosage units comprising 0.5 mg for the purpose of a daily administration, and 0.25 mg for the purpose of a twice-daily administration.

For an animal of 50 kg, the daily intake will have to be 2.5 mg/day. The kit will thus comprise dosage units comprising 2.5 mg for the purpose of a daily administration, and 1.25 mg for the purpose of a twice-daily administration.

The daily intake in two administrations may consist of the administration of two doses, each equivalent to half the daily dose, but not solely in this way. The twice-daily dose administered may for example be adjusted as a function of the time elapsed between two administrations. Thus, a dog that is treated at 8 o'clock in the morning and 6 o'clock in the evening may have 10/24 times the dose at 8 o'clock in the morning and 14/24 times the dose at 6 o'clock in the evening.

It goes without saying that the number of dosage units that may be administered daily to an animal of a given weight may be easily determined on the basis (i) of the daily dosage of torasemide that is suitable, per unit of weight, and (ii) of the weight of the animal. For example, it is possible to use a pharmaceutical kit suitable for an animal having a weight W1 and comprising dosage units each comprising a known amount of torasemide, and to administer, to an animal having a weight W2 greater than W1, the number of dosage units required to achieve, at least approximately, the daily dosage of torasemide that said animal of weight W2 must receive.

Of course, those skilled in the art understand that the expected effects of the treatment are obtained when the daily dose of torasemide actually received by the animal under consideration varies slightly compared with the optimal dose prescribed, especially for a long-term treatment. Thus, it should be understood that the numbers indicated in the application are intended to mean “approximately the number indicated”, a variation of 1, 2, 3, 4, 5, 6, 7, 8, 9 or 10% of the numerical value being acceptable, and the corresponding dosage being included in the invention.

For the purposes of the invention, the term “0.1 mg/kg” is interchangeable with the term “0.10 mg/kg”. Indeed, the upper limit daily dose of 0.1 mg/kg should be understood as being a dose of 0.10 mg/kg, this dose being included in the range indicated. The higher numbers, in particular the doses of 0.11, 0.12 or 0.13 mg/kg, are excluded from the range indicated.

For the purposes of the invention, the lower limit daily dose of 0.02 mg/kg is interchangeable with a dose of 0.020 mg/kg, this dose being included in the range indicated.

The dosages presented in the application are particularly suitable for dogs and cats. Naturally, those skilled in the art may adapt the dosage for other domestic animals, according to their general knowledge. According to the invention, the pharmaceutical kit is suitable for a long-term treatment. Thus, according to one preferred aspect of the invention, said kit comprises at least seven daily dosage units, or 14 units for a twice-daily administration.

According to another aspect of the invention, the kit may comprise seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine or thirty daily dosage units.

According to one particular aspect of the invention the composition comprising torasemide, for use thereof in the treatment of heart failure, is characterized in that it is administered to an animal during a treatment lasting at least seven days, and preferentially at least 14 days, at least 21 days, at least 28 days, at least 30 days, and more preferably for more than three months.

Preferably, the dosage units are suitable for oral administration.

According to one particular aspect of the invention, the veterinary pharmaceutical composition is administered orally. Thus, the “torasemide” active ingredient will be coupled to agents and excipients well known to those skilled in the art, which allow non-destruction and reabsorption of the active ingredient during its passage through the esophagus and the stomach of the animal having ingested it.

Those skilled in the art are aware of the various galenics for administering medicaments to animals orally, in particular to domestic animals, these galenics comprising in particular oral solutions, dragees, gel capsules, gels, emulsions, pastes, suspensions, sublingual films, tablets to be swallowed or crushed, tablets to be chewed, film-coated tablets, effervescent tablets, soluble tablets, dispersible tablets, orodispersible tablets, soft or hard capsules, soft capsules to be chewed, granulated material or granules to be dissolved or dispersed on the food, in drinking water or other appropriate carriers (in the form, for example, of a presentation form in pouches or of a pot with dose sachet), powders to be dissolved or dispersed on the food, in drinking water or other suitable carriers (in the form, for example, of a presentation form in pouches or of a pot with dose sachet), syrups, functional foods, liquids to be dispersed on the food and hydrogels.

According to one particular aspect of the invention, the torasemide is administered as a constituent of a complete animal food.

Preferably, these various galenics will exhibit a palatable aspect for the animal to be treated, i.e. the animal will by itself wish to swallow the galenic comprising the torasemide, according to the daily dosage previously defined.

According to one particular aspect of the invention, the dosage units or the composition are in a palatable form, in particular in the form of palatable tablets, either covered with a palatable film-coating or in a palatable form (for example, in the form of a titbit). According to one particular aspect of the invention, the dosage units or the composition are in the form of palatable chewable tablets.

The compositions according to the invention may be prepared by conventional methods of producing pharmaceutical oral forms using one or more physiologically acceptable functional excipients or carriers.

The oral compositions according to the invention may be obtained by means of one or more steps of intermediate preparation of the active agent, such as matrix encapsulation processes (dry granulation, wet granulation, extrusion, granulation by spray cooling or prilling, solvent evaporation spray drying, polymeric precipitation, solid lipid nanoparticles (SLNs), membrane encapsulation processes (film-coating assembly on nonpareils, coating of particles, ionotropic encapsulation, encapsulation by coacervation, liposomes, emulsion) and inclusion processes (absorption on porous solid supports, complexation in cyclodextrin, adsorption on ion exchange resins).

These pharmaceutical compositions may for example be prepared with excipients or carriers chosen from the following nonexhaustive list:
diluents, such as, for example, lactose, sucrose, glucose, dextrose and other sugars, microcrystalline cellulose and other cellulose-based derivatives, starches of various origins, calcium phosphate and derivatives thereof, calcium carbonates and bicarbonates, sodium carbonates and bicarbonates, potassium carbonates and bicarbonates, sodium glycine carbonate, sorbitol, mannitol, maltitol, xylitol, isomalt, and other polyols, glycine,
assembly or absorption supports (sugar, starch or cellulose nonpareils),
dry-process or wet-process binders, such as, for example, cellulose derivatives (ethylcellulose (EC), hydroxypropylmethylcellulose (HPMC), hydroxypropylmethylcellulose (HPC), methylcellulose (ME)), polyvinylpyrrolidone (PVP), pregelatinized starch, macrogols, polyethylene glycol (PEG), glyceryl palmitostearate, esters of glycerol and of behenic acid, gums (Arabic, acacia and tragacanth), gelatin, starch (in paste form), sugar (sucrose, glucose, sorbitol) solutions, maltodextrin,
disintegrants or disaggregating agents such as, for example, sodium starch glycolate, sodium croscarmellose, pregelatinized starch, starches of various origins, microcrystalline cellulose, sodium carboxymethyl starch, crosslinked polyvinylpyrrolidone (crosslinked PVP), alginic acid, effervescent pairs (combination of citric acid with an alkali metal carbonate or bicarbonate),
flow regulators, such as, for example, colloidal or precipitated silicas, which may be hydrophilic or hydrophobic, talc, starch, stearic acid,
lubricants such as, for example, talc, magnesium stearate, zinc stearate, calcium stearate, sodium stearate or aluminum stearate, sodium stearyl fumarate, mineral oils, hydrogenated vegetable oils, glyceryl palmitostearate, polyethylene glycol, carnauba wax, stearic acid, boric acid, sodium benzoate,
suspending agents such as, for example, sucrose, glucose syrups or sorbitol syrups, cellulose derivatives, hydrogenated fats, silicas, gums, alginates,
wetting agents or surfactants, such as, for example, sodium lauryl sulfate, polysorbate 80, lecithin, gum Arabic,
aqueous or nonaqueous carriers, such as, for example, water, hydrogenated or nonhydrogenated vegetable oils (olive oil, peanut oil), ethyl alcohol, glucose syrups or sorbitol syrups, glycerol, propylene glycol, polyethylene glycol, mineral oils (liquid petroleum jelly),
flavorings, such as, for example, natural meat or fish flavorings, synthetic flavorings, natural or synthetic aromatic compositions, brewer's yeasts,
sweeteners such as, for example, sucralose, aspartame, acesulfame potassium, neotame, alitame, cyclamate, sucrose, glucose, fructose, maltitol, sorbitol, xylitol, stevia and saccharin,
dyes such as, for example, iron oxides, titanium dioxide,
masking agents such as, for example, acidifying agents including citric acid, tartaric acid, malic acid, fumaric acid, succinic acid, adipic acid,
taste enhancers such as, for example, glutamates, guanylates, inosinates, maltol, ethyl maltol, glycine, L-leucine, lactic acid, thaumatin, neohesperidin,
coating (film-coating or sugar-coating) excipients, such as, for example, plasticizers (glycerol, propylene glycol, PEGs (macrogols), glyceryl triacetate, triethyl citrate, triethyl acetyl citrate, castor oil, tributyl citrate (insoluble), tributyl acetyl citrate, diethyl phthalate, film-forming agents (cellulose derivatives such as ethyl cellulose (EC), hydroxypropylmethylcellulose (HPMC), hydroxypropylmethylcellulose (HPC), methylcellulose (ME), cationic copolymers of acrylic acid and of methacrylic esters, copolymers of dimethylaminoethyl methacrylate and of neutral methacrylic esters, shellac, cellulose acetate phthalate (CAP), polyvinyl phthalic acetate (PVPA), HPMC phthalate (HPMCP) and anionic copolymers of acrylic acid and of methacrylic esters, cellulose acetate, neutral copolymers of acrylic and methacrylic esters,
polishes, such as carnauba wax,
fillers such as, for example, sugar, silicas, talc, liquid or pasty silicones, titanium dioxide,
antioxidants such as, for example, ascorbic acid, sodium and calcium ascorbates, 5,6-diacetyl-1-ascorbic acid, 6-palmityl-1-ascorbic acid, citric acid, potassium sodium and calcium citrates, tartaric acid, potassium sodium and sodium and potassium tartrates, butylhydroxyanisol, butylhydroxytoluol, octyl or dodecyl gallates, sodium, potassium or calcium lactates, lecithins, alpha-tocopherol, gamma-tocopherol and delta-tocopherol, all of the tocopherols constituting vitamin E,
preservatives such as, for example, sorbic, fumaric and benzoic acids, para-hydroxybenzoic acid esters, alcohol, benzyl alcohol, chlorocresol, sulfites, nitrates and nitrites.

In one particular mode of the invention, the tablets may be gastroresistant, or modified-release tablets.

According to one particular aspect, the oral solid forms may be dividable in order to facilitate a dosage suitable for the treatment of the animal, such as, for example, a scored tablet that can be broken into 2, 3, 4, 5, 6, 7 or 8 equivalent dose units.

According to another aspect of the invention, each dosage unit comprises torasemide in combination with at least one other active ingredient, in particular an active ingredient used in the treatment of heart failure.

According to another aspect of the invention, the veterinary pharmaceutical composition comprises torasemide in combination with at least one other active ingredient, in particular an active ingredient used in the treatment of heart failure.

The active ingredients conventionally used in the treatment of heart failure are the following:
angiotensin-converting enzyme inhibitors (ACEIs),
phosphodiesterase inhibitors,
aldosterone antagonists,
the combination of an ACEI with an aldosterone antagonist: benazepril+ spironolactone.

This active ingredient may in particular be chosen from the following active ingredients:
selective phosphodiesterase III (PDEIII) inhibitors, and in particular pimobendan, used at doses of between 0.1 mg/kg/day and 1 mg/kg/day;
ACEIs and in particular benazepril, enalapril, imidapril and ramipril, used according to the dosages well known to those skilled in the art (see http://www.cbip-vet.be/fr/texts/FCVOOOL1AL2o.php);
antihypertensives, and in particular aldosterone antagonists, preferentially spironolactone, used in particular at a dose of 2 mg/kg/day.

The following combinations may in particular be present in the veterinary pharmaceutical kit according to the invention, in the form of daily units:
torasemide and pimobendan;
torasemide and benazepril;
torasemide and enalapril;
torasemide and imidapril;
torasemide and ramipril;
torasemide and spironolactone;
torasemide and benazepril and spironolactone.

In particular, for a kit intended for an animal of 10 kg, the following dosage units may be present in the kits according to the invention:
0.5 mg of torasemide and 2.5 mg of pimobendan; or
0.5 mg of torasemide and 5 mg of benazepril; or
0.5 mg of torasemide and 20 mg of spironolactone.

The following combinations may in particular be present in the veterinary pharmaceutical composition according to the invention:
torasemide and pimobendan;
torasemide and benazepril;
torasemide and enalapril;
torasemide and imidapril;
torasemide and ramipril;
torasemide and spironolactone;
torasemide and benazepril and spironolactone.

In particular, for a pharmaceutical composition intended for an animal of 10 kg, the following combinations may be prepared:
0.5 mg of torasemide and 2.5 mg of pimobendan; or
0.5 mg of torasemide and 5 mg of benazepril; or
0.5 mg of torasemide and 20 mg of spironolactone.

The invention also relates to the veterinary pharmaceutical kit presented above, for use thereof in the treatment and/or prevention of heart failure in mammals, preferably in cats or dogs.

The invention also relates to the veterinary pharmaceutical composition presented above, for use thereof in the treatment and/or prevention of heart failure in mammals, preferably in cats or dogs. The composition will thus be administered to a cat or a dog.

The invention also relates to the veterinary pharmaceutical kit presented above, for use thereof in the treatment and/or prevention of heart failure in elderly dogs.

The invention also relates to a veterinary pharmaceutical composition as presented above, for use thereof in the treatment and/or prevention of heart failure in elderly dogs.

The term “elderly dog” is intended to mean a dog in the last quarter of its life, calculated according to its average life expectancy. Life expectancy varies according to the breed of the dog. For example, a Beagle usually lives between 12 and 15 years (average life expectancy 13.3 years) and a Scottish terrier usually lives to between 10 and 16 years old (average life expectancy of 12 years). In the Western world, the “mongrel dog” has an average life expectancy of 13.2 years. Thus, the term “elderly dog” is intended to mean, for a mongrel dog, a dog with an age above 9.9 years, in particular equal to or above 10 years.

The composition according to the invention is also more particularly intended for dogs of a breed having a predisposition to cardiac pathological conditions, such as dogs of the following breeds: Poodle, Chihuahua, Bichon, Yorkshire, Cavalier King Charles, Pekingese, Pinscher, Keeshond, Spaniels, English Springer Spaniel, Pomeranian, Basset, Beagle, Westie, Whippet, Terriers, Fox terrier, Yorkshire Terrier.

The composition may thus be administered to an elderly dog and/or to a dog of a breed predisposed to cardiac pathological conditions.

The invention also relates to the veterinary pharmaceutical kit presented above, for use thereof in the treatment and/or prevention of heart failure in an animal in the decompensation phase.

The invention also relates to the veterinary pharmaceutical composition presented above, for use thereof in the treatment and/or prevention of heart failure in an animal in the decompensation phase.

The invention also relates to the veterinary pharmaceutical kit presented above, for use thereof in the treatment and/or prevention of the occurrence of edema in an animal in the decompensation phase.

The invention also relates to the veterinary pharmaceutical composition presented above, for use thereof in the treatment and/or prevention of the occurrence of edema in an animal in the decompensation phase.

The invention also relates to the veterinary pharmaceutical kit presented above, for use thereof in maintaining the sodium balance in an animal suffering from a sodium imbalance.

The invention also relates to the veterinary pharmaceutical composition presented above, for use thereof in maintaining the sodium balance in an animal suffering from a sodium imbalance.

A sodium imbalance can be defined as abnormal sodium levels observed in the plasma and/or the urine of an animal. Those skilled in the art will easily find the reference “normal” values for each type of animal, and in particular for each breed of dog. In particular, the article by Laroute et al., 2005, gives normal values for urinary sodium clearance in female Beagles, and that by Bennett et al. (Australian Veterinary J., 2006) indicates the reference values for the fractional excretion of sodium in dogs of the Greyhound breed.

The amount of sodium present in the body of mammals regulates blood volume, blood pressure, and general osmotic equilibrium. Sodium is absorbed via the diet and its concentration into the body is regulated by the renin-angiotensin system. In the event of imbalance, of sodium concentrations that are too high or too low, the entire renin-angiotensin system is disrupted and the functioning of numerous organs is affected. The use of torasemide at low dose, less than 0.1 mg/kg/day, makes it possible to maintain a normal sodium balance in the body of an animal.

An animal suffering from sodium imbalance may in particular be an animal treated for heart failure.

The invention also relates to the following subjects:
A method for treating an animal suffering from heart failure, consisting in administering daily to this animal a composition comprising torasemide at a dosage of between 0.02 mg/kg and 0.1 mg/kg during a long-term treatment.
A method as described above, the composition comprising torasemide at a dosage of 0.05 mg/kg.
A method as described above, the composition being administered in two daily intakes.
A method as described above, the composition being administered during a treatment lasting at least seven days.
A method as described above, the composition being in the form of tablets, optionally palatable tablets.
A method as described above, the composition also comprising at least one second active ingredient chosen from selective phosphodiesterase III (PDEIII) inhibitors, ACE inhibitors and antihypertensives.
A method as described above, characterized in that the composition is administered to a dog or a cat, in particular to an elderly dog or to a dog of a breed predisposed to cardiac pathological conditions.
A method as described above, characterized in that the animal treated is in the decompensation phase.
A method as described above, making it possible to prevent and/or treat the occurrence of edema in an animal suffering from heart failure, in the decompensation phase.
A method intended for maintaining the sodium balance of the body in an animal suffering from a sodium imbalance, consisting in administering daily to this animal a composition comprising torasemide at a dosage of from 0.02 mg/kg to 0.1 mg/kg during a long-term treatment.
A method as described above, making it possible to prevent a sodium imbalance in an animal suffering from heart failure, consisting in administering daily to this animal a composition comprising torasemide at a dosage of from 0.02 mg/kg to 0.1 mg/kg during a long-term treatment.

### Examples

A pharmacokinetic/pharmacodynamic study aimed at exploring the dose-effect relationship of torasemide in dogs, and of comparing the effects of torasemide with the regular dose of furosemide (5 mg/kg), was carried out in female Beagle dogs. Several criteria were evaluated in order to determine the efficacy of this dosage:
urine volume
water consumption
urinary sodium clearance
urinary potassium clearance
osmolar clearance
free-water clearance
urinary fraction excretion of sodium
urinary fraction excretion of potassium.

Other criteria were evaluated in order to determine the safety of this dosage:
plasma creatinine
creatinine clearance
plasma urea
plasma aldosterone.

The experiment is carried out in the following way:
on day “D-7”, the 9 dogs undergo a clinical examination and the following measurements are carried out:
   in the urine: volume, pH, osmolarity, sodium, potassium;
   in the plasma: sodium, potassium, aldosterone.
the dogs are then divided up into 3 groups:
   3 dogs=group 1=torasemide 0.5 mg/kg/day
   3 dogs=group 2=torasemide 0.05 mg/kg/day
   3 dogs=group 3=furosemide 5 mg/kg/day.
the dogs are treated for 21 days from D7 to D28. The following measurements are carried out:
→urine: volume, pH, creatinine, osmolarity, sodium, potassium;
→plasma: sodium, potassium, urea, aldosterone.

### Example 1. Dose-Dependent Effect of Torasemide on Urinary Sodium Clearance

In this experiment, torasemide is tested at two single doses: 0.05 mg/kg or 0.5 mg/kg for a daily administration for three weeks. The continuous administration of diuretics causes a net deficit of sodium ions in the body, but renal compensation mechanisms adjust sodium excretion in line with sodium intake. This phenomenon is known as “diuretic braking”. This braking effect occurs in dogs in good health as soon as a single dose of 0.5 mg/kg of torasemide has been administered.

The reference value for urinary sodium clearance in female Beagles is 5.23±2.74 ml/h (Laroute et al., 2005).

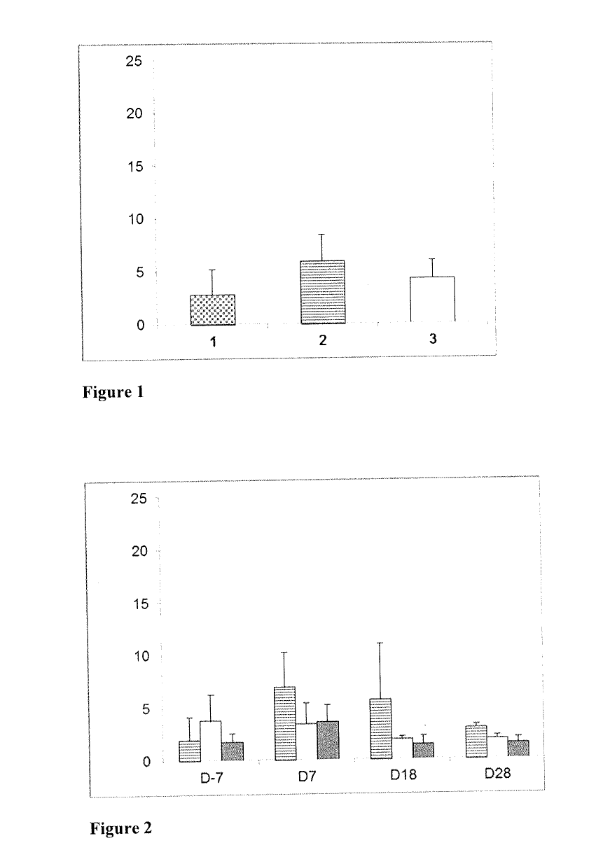

It was observed that, after administration of a single dose of torasemide, the overall effect on sodium clearance over the course of 24 hours of the low dose of torasemide (0.05 mg/kg) was higher than the overall effect of the high dose of torasemide (0.5 mg/kg) (FIG. 1).

The same result was obtained after repeated administrations of torasemide; it should be noted that the high dose of torasemide (and the regular dose of furosemide) had no net effect over the course of 24 hours on urinary sodium clearance, whereas the low dose of torasemide (0.5 mg/kg) had an effect that was clearly visible after the first day (D7) and the subsequent days (D18, D28) of treatment (FIG. 2).

These results strongly suggest that, in healthy dogs, a dose of 0.5 mg/kg of torasemide is not suitable and that the dose-effect relationship for torasemide is not an increasing monotonic curve, but instead an inverted U-shaped curve with an optimal dose located somewhere between 0.05 and 0.5 mg/kg/day of torasemide.

### Example 2. Dose-Dependent Effect of Torasemide Administration for Three Weeks in Healthy Dogs on Urine Volume and Water Consumption

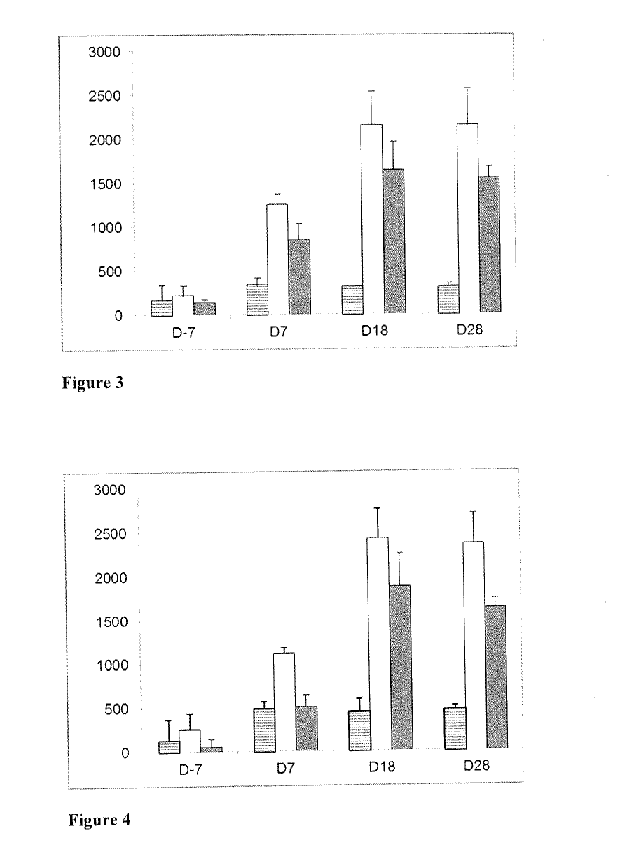

The urine volume observed at D-7, D7, D18 and D28 is presented in FIG. 3. The dogs treated with the low dose of torasemide (0.05 mg/kg/day) do not show any change in their urine volume. Conversely, the administration of 0.5 mg/kg/day of torasemide, or of furosemide (5 mg/kg/day), leads to a clear increase in urine volume as early as the first day of treatment (D7), this effect being maintained over the course of the three weeks of treatment.

The water consumption of the treated dogs, measured over a period of 24 h, is presented in FIG. 4. The dogs treated with the low dose of torasemide (0.05 mg/kg/day) show only a small change in their water absorption. Conversely, the administration of 0.5 mg/kg/day of torasemide, or of furosemide (5 mg/kg/day) leads to a clear increase in water consumption, especially after 11 days (D18) and 21 days (D28) of treatment.

### Example 3. Dose-Dependent Effect of Torasemide Administration for Three Weeks in Healthy Dogs on Urinary Potassium Clearance

The excretion of potassium ions in the urine of the treated dogs was measured over a period of 24 hours, and is presented in FIG. 5. The potassium ion excretion slightly increased whatever the treatment after a few days of treatment (D18 and D28). On the first day of treatment, the effect of the low-dose torasemide treatment is lower than with the other treatments.

### Example 4. Verification of the Innocuousness of a Treatment with Torasemide at 0.05 mg/kg/Day During a Long-Term Treatment

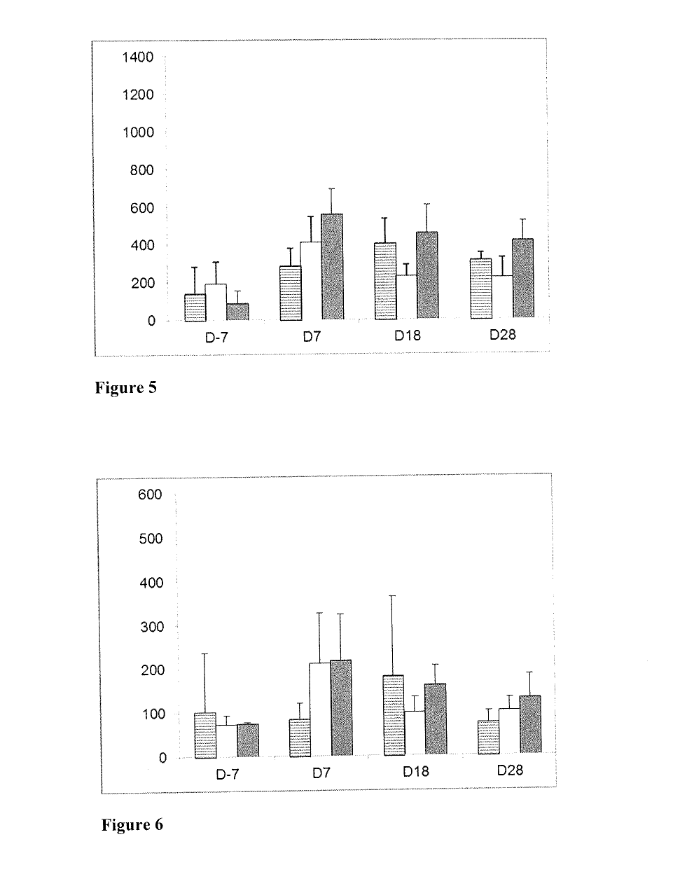

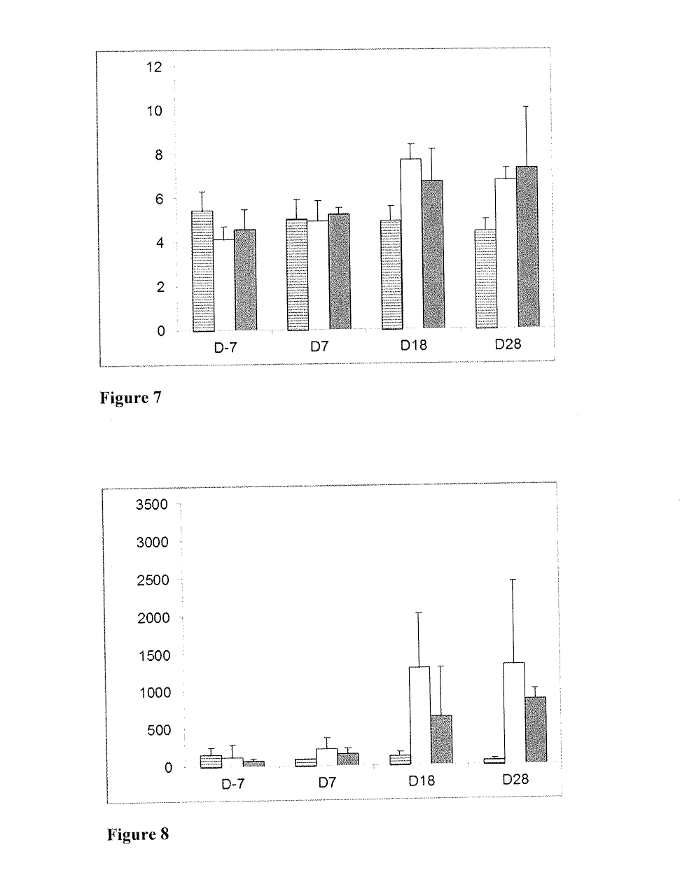

The following criteria are checked, 11 days and 21 days after the start of the treatment:
plasma creatinine (results not presented)
creatinine clearance (FIG. 6)
plasma urea (FIG. 7)
plasma aldosterone (FIG. 8).

The treatment has no effect on plasma creatinine concentration, whatever the day of the sample, nor on creatinine clearance (see FIG. 6).

Regarding the concentration of urea in the plasma, although high doses of torasemide and of furosemide lead to an increase in plasma urea concentration at D18 and D28, this harmful collateral effect is not observed during a treatment with a low dose of torasemide.

Regarding the aldosterone concentration in the plasma, although high doses of torasemide and of furosemide lead to an increase in plasma aldosterone concentration at D18 and D28, this harmful collateral effect is not observed during a treatment with a low dose of torasemide.

These results demonstrate that the main harmful effects observed during long-term treatments with torasemide disappear if the torasemide doses are below 0.1 mg/kg/day.

### Example 5. Dose-Dependent Effect on Torasemide Administration in Sick Dogs

In the experimental studies described above, carried out on healthy dogs, the modification of urinary sodium excretion by torasemide is evaluated by calculating urinary sodium clearance according to the following equation:

Urinary sodium concentration×total urine volume/plasma sodium concentration

A similar study is carried out in order to determine the optimal dose of torasemide in a clinical context, in sick dogs. However, in the clinical studies the clearance cannot be calculated since it would require the insertion of a urinary catheter over the course of several hours, which would lead to hospitalization of the animal, which would not be accepted by animal owners.

It is therefore necessary to be able to make a link between the clinical effect of torasemide which is observed “in the field” on sick dogs, and the effects of torasemide observed on urinary sodium excretion on healthy dogs.

One of the solutions for making the link between clinical and experimental is the calculation of the fractional excretion of sodium. This value is the ratio of the urinary sodium clearance to the urinary creatinine clearance. It requires assaying the creatinine in the plasma and in the urine, and also assaying the sodium in the plasma and the urine also, which is entirely envisionable “in the field”.

This fractional excretion of sodium (FENa) represents the percentage of sodium filtered by the kidney that is finally excreted in the urine. It is determined using equation 1 below:

$$FENa ( \% ) = \frac{{Na}_{urine} \times {Creatinine}_{plasma}}{{Na}_{plasma} \times {Creatinine}_{urine}} \times 100 \qquad Equation 1$$

where Naᵤᵣᵢₙₑ and Naₚₗₐₛₘₐ are the sodium concentrations in the urine and the plasma, respectively, and Creatinineₚₗₐₛₘₐ and Creatinineᵤᵣᵢₙₑ are the creatinine concentrations in the plasma and the urine, respectively. The fractional excretion of sodium is the sodium excretion divided by the glomerular filtration rate (GFR), the GFR being estimated by the creatinine clearance.

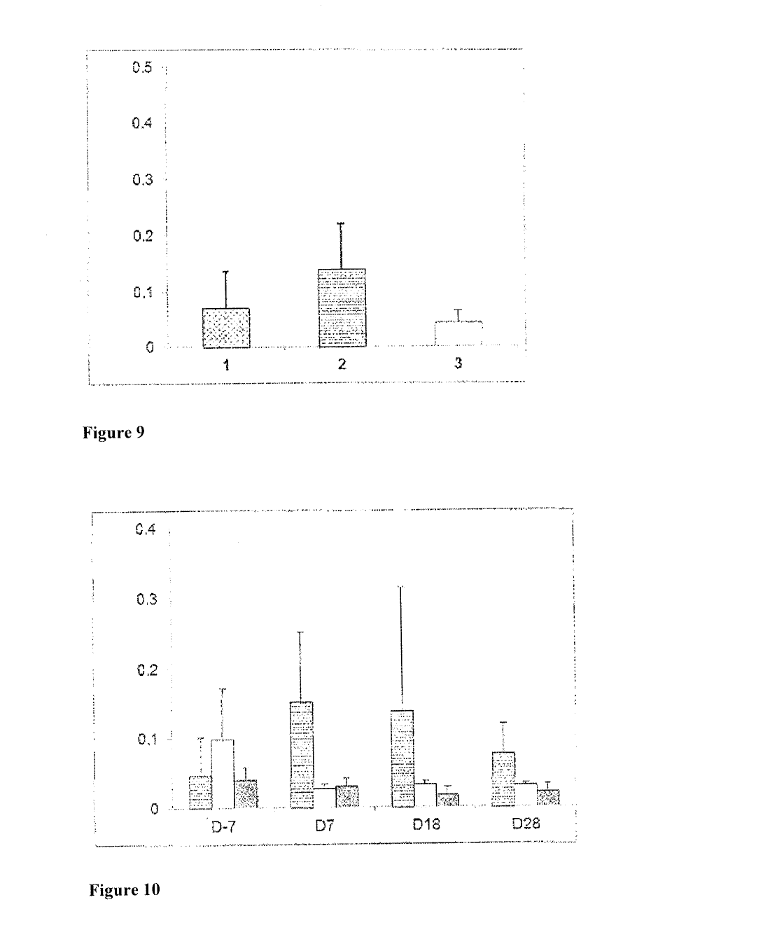

The fractional excretion of sodium was measured on healthy dogs after a single administration of torasemide at 0.05 mg/kg or 0.5 mg/kg (FIG. 9), and after repeated administrations of torasemide at 0.05 mg/kg/day, torasemide at 0.5 mg/kg/day or furosemide at 5 mg/kg/day for three weeks (FIG. 10).

The results obtained by estimating the fractional excretion of sodium are in agreement with those obtained by directly measuring the urinary sodium clearance, as presented in FIGS. 1 and 2. This measurement may thus be validly used to quantify the effect of the administration of a low dose of torasemide to dogs suffering from heart failure, in which it is not possible, from a practical point of view, to insert a urinary catheter for 24 hours.

### REFERENCES

### Patents

EP 2 514 421

### Non-Patents

Ghys A, Denef J, de Suray J M, Gerin M, Georges A, Delarge J, Willems J Pharmacological properties of the new potent diuretic torasemide in rats and dogs. Arzneimittel-Forschung [1985, 35(10):1520-1526]
Gerin M, Georges A, Delarge J, Willems J Pharmacological properties of the new potent diuretic torasemide in rats and dogs. Arzneimittel-Forschung [1985, 35(10):1520-1526]
Yasutomo Hori; Fumihiko Takusagawa; Hiromi Ikadai; Masami Uechi, Fumio Hoshi; Sei-ichi Higuchi. Effects of oral administration of furosemide and torsemide in healthy dogs. American Journal of Veterinary Research, October 2007, Vol. 68, No. 10, Pages 1058-1063
Uechi M, Matsuoka M, Kuwajima E, Kaneko T, Yamashita K, Fukushima U, Ishikawa Y. The effects of the loop diuretics furosemide and torasemide on diuresis in dogs and cats. J Vet Med Sci. 2003 October; 65(10):1057-61.
Caro-Vadillo A, Ynaraja-Ramirez E, Montoya-Alonso J A. Effect of torsemide on serum and urine electrolyte levels in dogs with congestive heart failure. Vet Rec. 2007 Jun. 16; 160(24):847-8.
Peddle G D, Singletary G E, Reynolds C A, Trafny D J, Machen M C, Oyama M A. Effect of torsemide and furosemide on clinical, laboratory, radiographic and quality of life variables in dogs with heart failure secondary to mitral valve disease. J Vet Cardiol. 2012 March; 14(1):253-9.
Laroute V, Chetboul V, Roche L, Maurey C, Costes G, Pouchelon J L, De La Farge F, Boussouf M, Lefebvre H P. Quantitative evaluation of renal function in healthy Beagle puppies and mature dogs. Res Vet Sci. 2005 October; 79(2):161-7.
Bennett S L, Abraham L A, Anderson G A, Holloway S A, Parry B W. Reference limits for urinary fractional excretion of electrolytes in adult non-racing Greyhound dogs. Aust Vet J. 2006 November; 84(11):393-7.

1-12. (canceled)

13. A method for treating an animal suffering from heart failure, consisting in administering daily to this animal a composition comprising torasemide at a dosage of between 0.02 mg/kg and 0.1 mg/kg during a long-term treatment.

14. The method of claim 13, wherein the composition comprises torasemide at a dosage of 0.05 mg/kg.

15. The method of claim 13, wherein the composition is administered in two daily intakes.

16. The method of claim 13, wherein the composition is administered during a treatment lasting at least seven days.

17. The method of claim 13, wherein the composition is under the form of tablets.

18. The method of claim 13, wherein the composition further comprises at least one second active ingredient chosen from the group consisting of: selective phosphodiesterase III (PDEIII) inhibitors, ACE inhibitors and antihypertensives.

19. The method of claim 13, wherein the animal is a dog or a cat.

20. The method of claim 19, wherein the animal is an elderly dog.

21. The method of claim 19, wherein the animal is a dog from a breed predisposed to cardiac pathological conditions.

22. The method of claim 13, wherein the treated animal is in the decompensation phase.

23. The method of claim 22, wherein the treatment makes it possible to prevent and/or treat the occurrence of edema in the treated animal.

24. A method for maintaining the sodium balance of the body in an animal suffering from a sodium imbalance, consisting in administering daily to said animal a composition comprising torasemide at a dosage of from 0.02 mg/kg to 0.1 mg/kg during a long-term treatment.

25. The method of claim 24, wherein the composition comprises torasemide at a dosage of 0.05 mg/kg.

26. The method of claim 24, wherein the composition is administered in two daily intakes.

27. The method of claim 24, wherein the composition is administered during a treatment lasting at least seven days.

28. The method of claim 24, wherein the composition is under the form of tablets.

29. The method of claim 24, wherein the composition further comprises at least one second active ingredient chosen from the group consisting of: selective phosphodiesterase III (PDEIII) inhibitors, ACE inhibitors and antihypertensives.

30. The method of claim 24, wherein the animal is a dog or a cat.

31. The method of claim 30, wherein the animal is an elderly dog.

32. The method of claim 30, wherein the animal is a dog from a breed predisposed to cardiac pathological conditions.
